(19) United States
(12) Patent Application Publication
(10) Pub. No.: US 2024/0053336 A1
(43) Pub. Date: Feb. 15, 2024

(54) ANTI-MICROBIAL ANTIBODY SIGNATURES OF INFLAMMATORY BOWEL DISEASE AND USES THEREOF

(71) Applicants: Arizona Board of Regents on Behalf of Arizona State University, Scottsdale, AZ (US); Mayo Foundation for Medical Education & Research, Rochester, MN (US)

(72) Inventors: Mahasish SHOME, Tempe, AZ (US); Lusheng SONG, Tempe, AZ (US); Yunro CHUNG, Chandler, AZ (US); Jonathan LEIGHTON, Scottsdale, AZ (US); Joshua LABAER, Chandler, AZ (US); Ji QIU, Chandler, AZ (US)

(21) Appl. No.: 18/448,685

(22) Filed: Aug. 11, 2023

Related U.S. Application Data
(60) Provisional application No. 63/397,799, filed on Aug. 12, 2022.

(51) Int. Cl. G01N 33/569; G01N 33/68

(52) U.S. Cl. CPC G01N 33/56911; G01N 33/686; G01N 2800/065

(57) ABSTRACT

The present invention relates anti-microbial antibody signatures of inflammatory bowel disease and their use in early and accurate diagnosis of disease including ulcerative colitis (UC) and Crohn's disease (CD).

### CROSS-REFERENCE TO RELATED APPLICATIONS

This application claims the benefit of U.S. Provisional Application No. 63/397,799, entitled “Anti-Microbial Antibody Signatures of Inflammatory Bowel Disease and Uses Thereof,” which was filed Aug. 12, 2022, the entire disclosure of which is hereby incorporated herein by this reference.

### FIELD OF THE INVENTION

The invention relates to anti-microbial antibody signatures of inflammatory bowel disease and their use in early and accurate diagnosis of disease.

### BACKGROUND OF THE INVENTION

Inflammatory bowel disease (IBD) represents a group of intestinal disorders that causes chronic inflammation in the digestive tract. The two main clinical phenotypes are ulcerative colitis (UC) and Crohn's disease (CD). The public health burden of IBD is rising globally. Early and accurate diagnosis is key to reducing this burden. Gastroenterologists often use a combination of relatively invasive procedures, like ileocolonoscopy with biopsy for diagnosis, and to determine the disease extent and activity. There is a need for serological biomarkers that can reveal the disease state non-invasively.

### SUMMARY OF THE INVENTION

In some aspects, the disclosure concerns an antibody panel for diagnosing a subject with inflammatory bowel disease (IBD). The antibody panel described herein differentiates IBD from irritable bowl syndrome in a subject exhibit gastrointestinal symptoms. The antibody panel comprising at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, SP_1992, BILF2, CK_flgG, A4-Fla2, BVRF2, and UL139. In some embodiments, the inflammatory bowel disease is Crohn's disease, the antibody panel comprises at least at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2. In certain embodiments, the antibody panel comprises HP_0115, CK_LafA, CK_LafA.1, VC_flaD, VC_flaB, VC_flaE, and VC_flaA.

Some embodiments have an antibody panel comprising BVU_0562, SP_1992, PMI_RS06815, and SF_Lpp. Certain antibody panels comprise HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2.

In some embodiments, the inflammatory bowel disease is ulcerative colitis (UC), and the antibody panel comprises at least at least one antigen selected from the group consisting of: CK_flgG, A4-Fla2, BVRF2, and UL139. In certain embodiments, the antibody panel comprises CK_flgG, A4-Fla2, BVRF2, and UL139. In some implementations, the antibody panel can be used to distinguish between Crohn's disease (CD) and UC.

Other aspects of the disclosure concern methods of diagnosing IBD in a subject with gastrointestinal distress, the method comprising: (i) providing a biofluid sample from the subject with gastrointestinal distress; (ii) contacting the biofluid sample with at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, S P 1992, BILF2, CK_flgG, A4-Fla2, BVRF2, and UL139; and (iii) determining if the biofluid sample comprises an antibody against the at least one antigen, wherein the presence of the antibody against the at least one antigen diagnoses the subject with gastrointestinal distress with an inflammatory bowel disease. In some embodiments, the biofluid sample is blood or serum. In certain embodiments, the biofluid sample is blood.

In some embodiments, the biofluid sample is contacted with HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2, wherein the presence of antibodies against HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2 diagnoses the subject with gastrointestinal distress with CD. In certain embodiments, the biofluid sample is in contact with HP_0115, CK_LafA, CK_LafA.1, VC_flaD, VC_flaB, VC_flaE, and VC_flaA, wherein the presence of HP_0115, CK_LafA, CK_LafA.1, VC_flaD, VC_flaB, VC_flaE, and VC_flaA diagnoses the subject with gastrointestinal distress with CD instead of UC. In other embodiments, the antibody panel comprises BVU_0562, SP_1992, PMI_RS06815, and SF_Lpp, wherein the presence of BVU_0562, SP_1992, PMI_RS06815, and SF_Lpp diagnoses the subject with gastrointestinal distress with CD instead of UC.

In some embodiments, the antibody panel comprises HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2, wherein the presence of HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2 diagnoses the subject with gastrointestinal distress with CD instead of UC.

In other embodiments, the biofluid sample is contacted with CK_flgG, A4-Fla2, BVRF2, and UL139, wherein the presence of antibodies against CK_flgG, A4-Fla2, BVRF2, and UL139 diagnoses the subject with gastrointestinal distress with UC.

Yet other aspects of the disclosure concern methods of distinguishing the cause of gastrointestinal distress in a subject, the method comprising: (i) providing a biofluid sample from a subject with gastrointestinal distress; (ii) contacting the biofluid sample with at least one antigen selected from the group consisting of: SACOL2509, SACOL2511, SACOL2476, SPy 2009, HI_null, HI_oapA, SP_1479, SACOL1868, SACOL2509, HI_oapA, SP_0366, SP_0346, SP_0336, SP_1479, SP_0377, and SACOL2194; (iii) determining the biofluid sample comprises an antibody against the at least one antigen, wherein the presence of the antibody against the at least one antigen diagnoses the subject with gastrointestinal distress with an inflammatory bowel disease. In some embodiments, the biofluid sample is blood or serum. In certain embodiments, the biofluid sample is blood.

In some embodiments, the biofluid sample is contact with SACOL2509, SACOL2511, SACOL2476, SPy 2009, HI_null, HI_oapA, and SP_1479, the presence of antibodies against SACOL2509, SACOL2511, SACOL2476, SPy 2009, HI_null, HI_oapA, and SP_1479 diagnoses the subject with gastrointestinal distress with CD instead of UC. In other embodiments, the biofluid sample is contact with SACOL1868, SACOL2509, HI_oapA, SP_0366, SP_0346, SP_0336, SP_1479, SP_0377, and SACOL2194, the presence of antibodies against SACOL1868, SACOL2509, HI_oapA, SP_0366, SP_0346, SP_0336, SP_1479, SP_0377, and SACOL2194 diagnoses the subject with gastrointestinal distress with UC instead of CD.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B depict the phylogenetic tree of microbes studied with their corresponding number of proteins analyzed. FIG. 1A shows 50 species of bacteria with 1173 proteins were segregated into 6 phyla. FIG. 1B shows 33 species of viruses with 397 proteins were segregated into 10 phyla.

FIGS. 2A and 2B depict, in accordance with certain embodiments, the sequence homology of target antigens of validated antibodies and overlap of antibodies among Crohn's disease and ulcerative colitis. FIG. 2A depicts a heatmap showing sequence homology among target antigens for antibodies with validated performance of ≥14% sensitivity at 96% specificity comparing Crohn's disease (CD) patients with healthy controls. FIG. 2B shows that CD IgG, ulcerative colitis (UC) IgG, CD IgA and UC IgG represent the overlap of anti-microbial antibodies of IgG and IgA isotypes in CD and UC patients with ≥14% sensitivity at 96% specificity against healthy controls in the discovery set.

FIGS. 3A-3C depict, in accordance with certain embodiments, the receiver operating characteristic curves to discriminate Crohn's disease, ulcerative colitis, and healthy controls. FIG. 3A: Receiver operating characteristic (ROC) curve for Crohn's disease (CD) vs healthy controls. Area under the curve (AUC) values of novel anti-flagellin antibodies (HP_0115, CK_LafA, CK_LafA.1, VC_flaD, VC_flaB, VC_flaE, VC_flaA) and anti-non-flagellin antibodies (BVU_0562, SP_1992, PMI_RS06815, SF_Lpp) was 0.73 and 0.75, respectively. The AUC value obtained with a combination of novel anti-flagellin and anti-non-flagellin antibodies was 0.81; FIG. 3B: ROC curve for ulcerative colitis (UC) vs healthy controls. The AUC value obtained with a combination of 7 markers was 0.87; FIG. 3C: ROC curve for CD vs UC. The AUC value obtained with a combination of 7 markers was 0.82.

FIG. 4 depicts, in accordance with certain embodiments, a Spearman's rank correlation coefficient heatmap of anti-microbial antibodies and autoantibodies in Crohn's disease patients. The names of anti-microbial antibodies are colored in blue while autoantibodies are colored in black.

FIG. 5 depicts, in accordance with certain embodiments, comparisons of total number of antibodies in healthy controls, CD and UC at the bacterial species level. The number of proteins displayed on the microbial protein arrays for each species is shown in parenthesis. The statistical significance of the difference in seroprevalence between groups were calculated using Chi-squared test, *P<0.05, **P<0.01.

### DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “false positive” refers a test result which incorrectly indicates that a particular condition or attribute is present. Accordingly, the “false positive indicator”, in some aspects, refers to a biomarker that indicates the corresponding positive test result incorrectly indicates the presence of a particular condition or attribute, for example, cancer or an autoimmune condition.

Inflammatory bowel disease (IBD) is caused by a combination of genetic predisposition, faulty immune responses, and environmental factors. The interaction of microbes with the gut mucosa in a genetically susceptible individual and the corresponding immune response play a pivotal role in the initiation and progression of IBD. After birth, a limited diversity microbial community develops into a complex community due to the influence of diet and environmental factors. During the second or third decade of life, a dysbiosis is observed in IBD patients which leads to an imbalance between commensal and potentially pathogenic microorganisms. The healthy gut microbiota predominately comprises Firmicutes and Bacteroidetes, and to a lesser extent, Actinobacteria and Proteobacteria. In IBD, dysbiosis is observed with reduced abundance of Firmicutes and either higher or similar abundance of Proteobacteria. Besides compositional changes, genetic alterations also contribute to gut dysbiosis that leads to disease initiation and progression. For example, NOD2 variants were found in 20%-40% of European and American Crohn's disease (CD) patients. NOD2 encodes an intracellular receptor for the bacterial peptidoglycan muramyl dipeptide, which helps maintain the balance of commensal bacterial flora.

Immune response to microbes results in the production of antibodies to microbial antigens. Anti-Saccharomyces cerevisiae antibodies (ASCA) are associated with CD patients, with sensitivities and specificities ranging between 55% to 65% and 80% to 95%, respectively. Perinuclear antineutrophil cytoplasmic antibodies (pANCA) are associated with ulcerative colitis (UC) patients, with sensitivities and specificities ranging between 50% to 71% and 75% to 98%, respectively. Outer membrane protein of Escherichia coli (OmpC) and flagellin (CBir1) antibodies are prevalent in CD patients, with prevalence ranging between 24%-55% and 50%-56%. The number and response magnitude of anti-microbial antibodies have previously been shown to indicate the presence of IBD, its severity and its clinical course; however, the clinical utility of available antibodies in diagnosis and clinical management of IBD patients has been limited. The techniques used to discover the known anti-microbial antibodies associated with IBD are of low throughput and have only been applied to test on small number of candidate microorganisms or microbial antigens.

As shown in the examples below, an innovative protein microarray technology, namely Nucleic Acid Programmable Protein Array, was used to conduct a large-scale comparative profiling of anti-microbial antibodies in CD and UC patients and healthy controls. 1570 microbial proteins from the microbial protein collection (DNASU.org) from 50 bacteria and 33 viruses were selected based on preliminary studies and review of the literature, and they were displayed on microarrays and probed against 100 CD, 100 UC and 100 healthy control serum samples.

The microbiomic study performed in the Examples identified antibody signatures that can aid in the accurate diagnosis of IBD. Antibody responses to novel non-flagellin antigens with elevated prevalence in CD patients compared with healthy controls were identified. Many anti-microbial antibodies with lower prevalence in UC patients relative to healthy controls were also identified. The antibody panels disclosed herein could distinguish CD vs control, UC vs control and CD vs UC with AUCs of 0.81, 0.87, and 0.82, respectively.

This is an improvement from previously disclosed antibody panels. Lichtenstein et al. (Lichtenstein et al., “Combination of genetic and quantitative serological immune markers are associated with complicated Crohn's disease behavior.” Inflamm Bowel Dis, 2011, 17: 2488-2496) reported an integrated serological (ASCA-IgA, ASCA-IgG, anti-OmpC, anti-CBir1, anti-I2, pANCA) and genetic (SNP8, SNP12, SNP13) marker panel with an AUC of 0.80 to distinguish CD vs control. A panel of serological markers (ASCA-IgA, ASCA-IgG, ANCA, pANCA, OmpC, and CBir1) built by Plevy et al. (Plevy et al., “Combined serological, genetic, and inflammatory markers differentiate non-IBD, Crohn's disease, and ulcerative colitis patients.” Inflamm Bowel Dis, 2013, 19: 1139-1148) yields an AUC of 0.78 to distinguish CD vs UC. The antibody panels disclosed herein have comparable or better performance in IBD diagnosis or distinguishing CD from UC subtypes. A stronger anti-microbial antibody response with more aggressive disease in both CD and UC patients. Additionally, the anti-microbial antibodies and autoantibodies have different reactivity patterns in CD patients.

The results in the Examples also provide interesting insight into its pathogenesis. Antibody responses to proteins from Bacteroides vulgatus, Proteus mirabilis, Shigella flexneri and Streptococcus pneumoniae were elevated in CD patients. B. vulgatus has been reported to induce colitis in IBD-susceptible mice. P. mirabilis in gut can induce inflammation in cells and a colitis mouse model and has been associated with CD pathogenesis. Thus, the results in the Examples suggest that B. vulgatus and P. mirabilis may also play a role in human CD development. Reduced antibody responses was observed in UC patients to several genera of the Firmicutes phylum including Parvimonas micra, Streptococcus pyogenes, S. aureus, which were often reduced in abundance in UC patients' gut microbiota. For several genera belonging to Proteobacteria phylum, such as Haemophilus influenzae, Helibacter pylori, Klebsiella oxytoca, overall reduced antibody responses were observed; however, their abundance in the gut microbiota of UC patients has been reported to be either increased or remained the same compared with healthy controls.

Beyond exposure alone, antibody response requires functional immunological interaction between a microorganism and the host; however, anti-microbial antibodies by themselves do not prove causality. As such, source microorganisms whose antibodies show significant changes between IBD patients and healthy controls warrant future confirmation and functional assessment in causing IBD. A4-Fla2 flagellin included in the study showed IBD-specific prevalence with performance similar to that reported in the literature. Several antibodies to flagellins with higher prevalence in CD patients relative to healthy controls were also identified.

Previous studies mostly focused on antibodies with higher prevalence in IBD patients. The unbiased data-driven approach revealed the existence of many anti-microbial antibodies with higher prevalence in healthy controls relative to CD and especially UC patients. The reduction observed in CD and UC patients may be attributed to the dysbiosis and reduced diversity of gut microbiota in CD and UC patients. It is also possible that the reduction in anti-microbial antibodies in some CD and UC patients was in part because of immunosuppressive therapies they received. The greater number of antibodies having high prevalence in CD patients compared with UC patients indicates stronger anti-microbial humoral immunity in CD than in UC, which is consistent with reports in the literature that most known anti-microbial antibodies, such as ASCA, anti-OmpC, anti-Cbir1, and anti-I2, had higher prevalence in CD patients than in UC patients. This agreement, together with comparable performance of anti-flagellin antibodies in this study and that reported in the literature, suggests that the results reflect the microbial association of IBD etiopathology. However, the use of samples from patients with established disease and the lack of information on immunosuppressive therapies of these patients limited the interpretation of the results.

The association between anti-microbial antibody prevalence and various disease classifications was studied based on Montreal classification and surgery history, and more antibodies were found with significantly higher prevalence in patients with more aggressive disease behaviors relative to those with milder disease behavior. More antibodies with significantly higher prevalence in colonic CD patients relative to those in ileal CD patients were also found this the analysis. These results were consistent with previous reports that increasing diversity and magnitude of anti-microbial immune response was correlated with increased frequency of penetrating and/or structuring disease behavior. It is known that the colon has a microbial density of 1011-1012 anaerobic bacteria/gram while the ileum is colonized by 107-108 anaerobic bacteria/gram. Kleessen et al. (Kleessen et al., Mucosal and invading bacteria in patients with inflammatory bowel disease compared with controls. Scand J Gastroenterol, 2002, 37: 1034-1041) found higher percentage of bacterial invasion of mucosa in colon compared to ileum. CD patients requiring surgery usually had more severe disease compared with those who did not need surgery. Stronger anti-microbial immune response in patients with severe CD or UC suggests a higher abundance of the source microorganisms for the target antigens of the differential antibodies and/or a stronger more conducive immune microenvironment at the disease site in severe disease.

Both autoantibodies and anti-microbial antibodies associated with IBD have been reported. One popular hypothesis for the autoantibody elicitation is molecular mimicry, where anti-microbial antibodies cross react with human proteins. However, minimal correlation was found between the anti-microbial antibodies and the autoantibody profiles in the same set of CD samples. The lack of correlation suggests that IBD-specific autoantibodies and anti-microbial antibodies are elicited independently through different underlying mechanisms, and cross-reactivity may play less of a role in eliciting CD-associated autoantibodies. The breakdown of immune tolerance to human proteins might have occurred due to the damaged gut epithelial cells and the faulty immunological microenvironment partly caused by microbial infections. In addition, the elicitation of autoantibodies may be associated with the infections of multiple microorganisms, and the correlation with individual anti-microbial antibodies may not be great.

Strengths of the study include the broadest analysis to date of IgG and IgA antibodies against individual antigens from many different microorganisms in both CD and UC patients and the use of a two-stage approach with discovery and independent validation of antibody markers. There are some limitations to the study. Except for a few microbes, the number of proteins studied for each species is small, which might limit the interpretation of antibody response in IBD at the species level. Furthermore, many samples used in studies were collected from patients with established disease.

Accordingly, disclosed herein are anti-microbial antibody signatures of IBD, in particular CD and UC. These anti-microbial antibody signatures aid the early detection and diagnosis of IBD and can distinguish between IBD and irritable bowls syndrome (IBS), which is not an inflammatory condition. The anti-microbial antibody signatures of CD are antibodies against the antigens of Bacteroidetes vulgatus (BVU_0562) and Streptococcus pneumoniae (SP_1992). The levels of these antibodies were elevated in CD patients relative to healthy controls. The anti-microbial antibody signatures of UC are antibodies against the antigen of Streptococcus pyogenes (SPy 2009). The levels of these antibodies were found to be elevated in healthy controls relative to UC patients.

Also disclosed are antibody panels developed using these anti-microbial antibody signatures of IBD. Patients with severe disease had higher prevalence of anti-microbial antibodies. There was minimal correlation among the occurrence of autoantibodies and anti-microbial antibodies in CD patients. Subgroup analysis revealed that penetrating CD behavior, colonic CD location, CD patients with a history of surgery, and extensive UC exhibited highest antibody prevalence among all patients.

In some embodiments, the antibody panel for diagnosing a subject with IBD comprises at least one antigen, at least two antigens, at least three antigens, at least four antigens, or at least five antigens selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, S P 1992, BILF2, CK_flgG, A4-Fla2, BVRF2, and UL139. Some embodiments have an antibody panel comprising BVU_0562, SP_1992, PMI_RS06815, and SF_Lpp. Certain antibody panels comprise HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2.

Where the IBD is Crohn's disease, the antibody panel comprises at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2. In certain embodiments, the antibody panel for diagnosing a subject with CD comprises HP_0115, CK_LafA, CK_LafA.1, VC_flaD, VC_flaB, VC_flaE, and VC_flaA.

Where the IBD is ulcerative colitis, the antibody panel comprises at least one antigen selected from the group consisting of: CK_flgG, A4-Fla2, BVRF2, and UL139. In certain embodiments, the antibody panel comprises CK_flgG, A4-Fla2, BVRF2, and UL139.

In some embodiments, the antibody panel may be associated with an array or an enzyme-linked immunosorbent assay (ELISA). In some embodiments, the binding of the antibody to the antigen is detected using a secondary antibody, capable of binding to the antibody of interest, linked to a colorimetric detection system such as fluorescent dyes or enzyme substrate that generate a chemiluminescent signal. In some embodiments, the antigen is immobilized on the surface of a substrate using a coupling agent. The biofluid sample is then contacted with the antigen containing substrate. After contact any unattached material may be washed away from the panel.

Methods of diagnosing IBD in a subject with gastrointestinal distress are also disclosed. In one aspect, the method comprises: (i) providing a biofluid sample from the subject with gastrointestinal distress; (ii) contacting the biofluid sample with at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, S P 1992, BILF2, CK_flgG, A4-Fla2, BVRF2, and UL139; and (iii) determining if the biofluid sample comprises an antibody against the at least one antigen, wherein the presence of the antibody against the at least one antigen diagnoses the subject with gastrointestinal distress with IBD. In some embodiments, the biofluid sample is blood or serum. In certain embodiments, the biofluid sample is blood.

In some embodiments, the biofluid sample is contacted with HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2, wherein the presence of antibodies against HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2 diagnoses the subject with gastrointestinal distress with CD. In certain embodiments, the biofluid sample is in contact with HP_0115, CK_LafA, CK_LafA.1, VC_flaD, VC_flaB, VC_flaE, and VC_flaA, wherein the presence of HP_0115, CK_LafA, CK_LafA.1, VC_flaD, VC_flaB, VC_flaE, and VC_flaA diagnoses the subject with gastrointestinal distress with CD instead of UC. In other embodiments, the antibody panel comprises BVU_0562, SP_1992, PMI_RS06815, and SF_Lpp, wherein the presence of BVU_0562, SP_1992, PMI_RS06815, and SF_Lpp diagnoses the subject with gastrointestinal distress with CD instead of UC.

In some embodiments, the antibody panel comprises HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2, wherein the presence of HP_0115, BVU_0562, CK_LafA, CK_LafA.1, A4-Fla2, PMI_RS06815, VC_flaD, VC_flaB, VC_flaE, VC_flaA, SF_Lpp, S P 1992, and BILF2 diagnoses the subject with gastrointestinal distress with CD instead of UC.

In other embodiments, the biofluid sample is contacted with CK_flgG, A4-Fla2, BVRF2, and UL139, wherein the presence of antibodies against CK_flgG, A4-Fla2, BVRF2, and UL139 diagnoses the subject with gastrointestinal distress with UC.

Yet other aspects of the disclosure concern methods of distinguishing the cause of gastrointestinal distress in a subject. The method comprises: (i) providing a biofluid sample from a subject with gastrointestinal distress; (ii) contacting the biofluid sample with at least one antigen selected from the group consisting of: SACOL2509, SACOL2511, SACOL2476, SPy 2009, HI_null, HI_oapA, SP_1479, SACOL1868, SACOL2509, HI_oapA, SP_0366, SP_0346, SP_0336, SP_1479, SP_0377, and SACOL2194; (iii) determining the biofluid sample comprises an antibody against the at least one antigen, wherein the presence of the antibody against the at least one antigen diagnoses the subject with gastrointestinal distress with an inflammatory bowel disease. In some embodiments, the biofluid sample is blood or serum. In certain embodiments, the biofluid sample is blood.

In some embodiments, the biofluid sample is contact with SACOL2509, SACOL2511, SACOL2476, SPy 2009, HI_null, HI_oapA, and SP_1479, the presence of antibodies against SACOL2509, SACOL2511, SACOL2476, SPy 2009, HI_null, HI_oapA, and SP_1479 diagnoses the subject with gastrointestinal distress with CD instead of UC. In other embodiments, the biofluid sample is contact with SACOL1868, SACOL2509, HI_oapA, SP_0366, SP_0346, SP_0336, SP_1479, SP_0377, and SACOL2194, the presence of antibodies against SACOL1868, SACOL2509, HI_oapA, SP_0366, SP_0346, SP_0336, SP_1479, SP_0377, and SACOL2194 diagnoses the subject with gastrointestinal distress with UC instead of CD.

### EXAMPLES

The present invention is further illustrated by the following examples that should not be construed as limiting. The contents of all references, patents, and published patent applications cited throughout this application, as well as the Figures, are incorporated herein by reference in their entirety for all purposes.

### I. Anti-Microbial Antibody Profiling in IBD on Microbial Protein Arrays

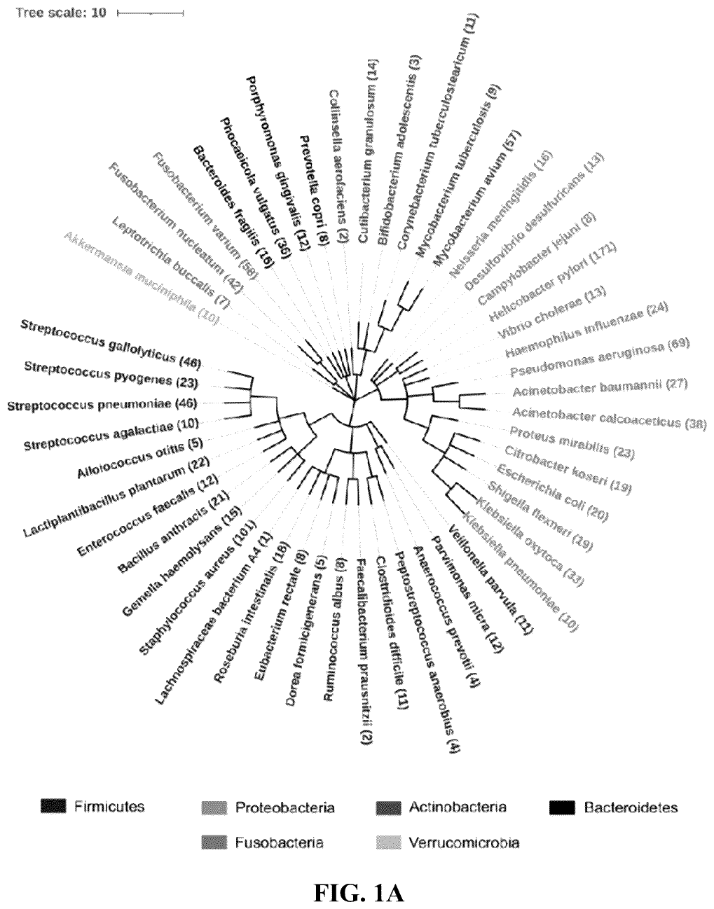

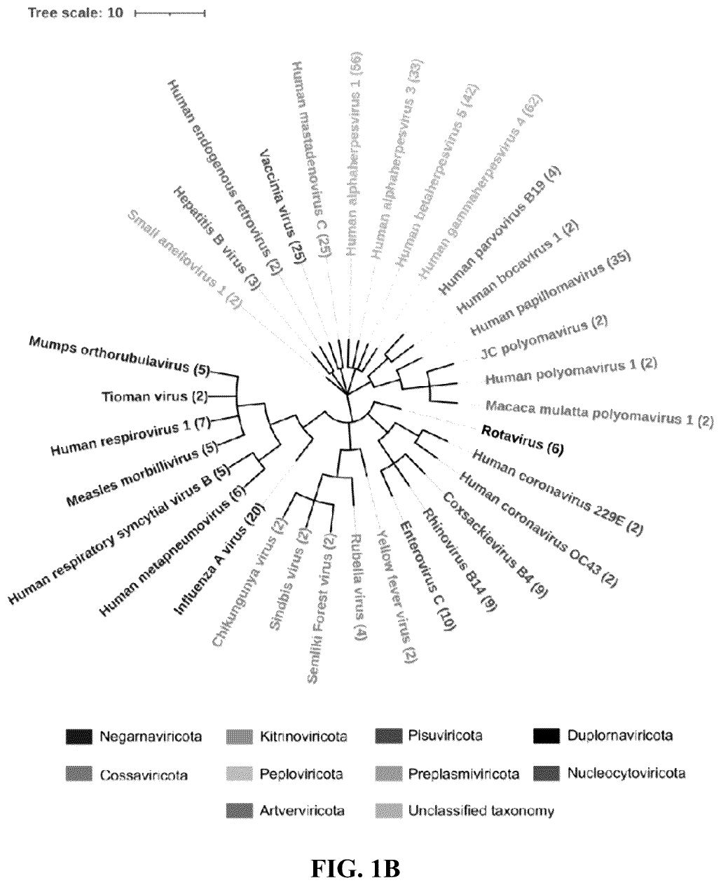

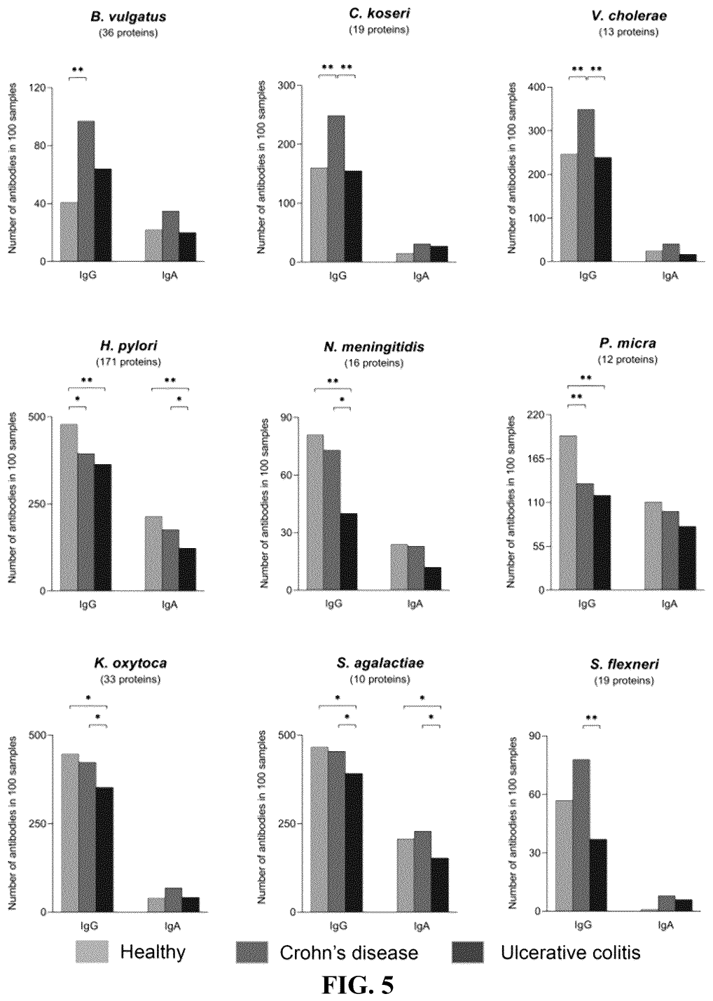

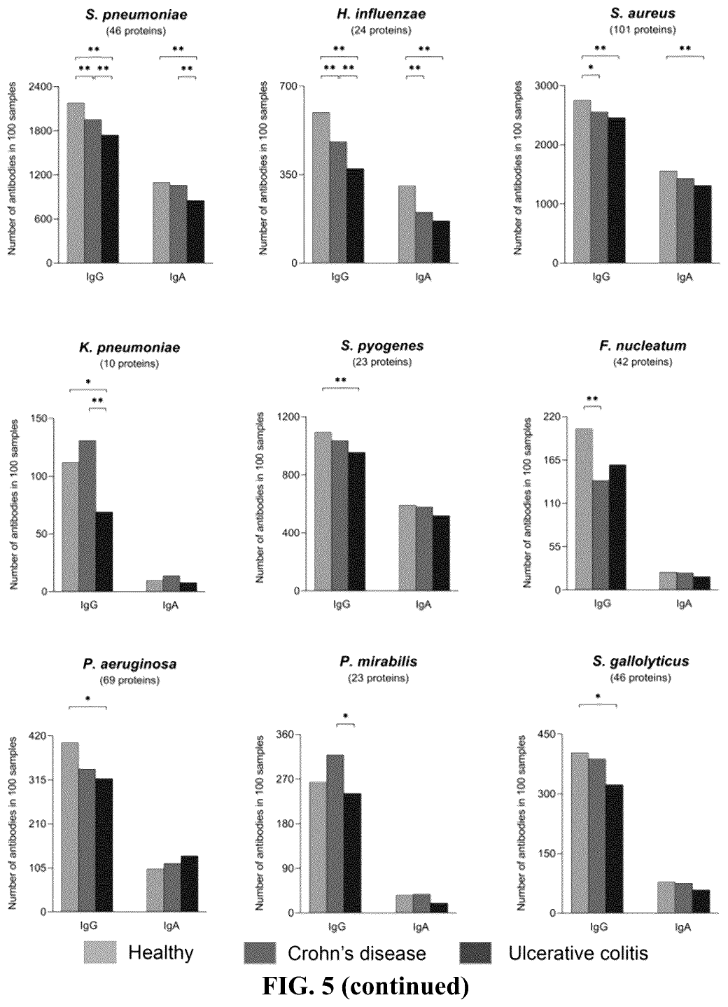

IgG and IgA anti-microbial antibody profiles of 100 CD and 100 UC patients and 100 age-gender matched healthy controls (Table 1) against 1570 microbial antigens including 1173 antigens from 50 different bacteria and 397 antigens from 33 different viruses using the protein microarray platform (FIG. 1, Table 2). This study provided a representative overview of the anti-microbial antibody response in IBD patients (FIG. 5). The numbers of IgG antibodies against bacterial proteins from Bacteroidetes vulgatus (B. vulgatus) and Citrobacter koseri (C. koseri) were significantly higher in CD patients compared with those in healthy controls (Chi-square test, P<0.01) (FIG. 5). On the contrary, the numbers of IgG antibodies against proteins from several bacteria, such as Streptococcus pneumoniae (S. pneumoniae), Haemophilus influenza (H. influenzae), Staphylococcus aureus (S. aureus), Helicobacter pylori (H. pylori) and Parvimonas micra (P. micra) were significantly lower in CD and UC patients compared with those in healthy controls (Chi-square test, P<0.05) (FIG. 5). Overall, fewer IgA anti-microbial antibodies were found than IgG antibodies. The numbers of IgA antibodies against S. pneumoniae, H. influenzae, S. aureus, and H. pylori were significantly lower in UC patients compared with those in healthy controls (Chi-square test, P<0.01). On the other hand, anti-viral IgG and IgA antibodies showed heterogenous prevalence with no clear trend of differences among CD, UC, healthy controls. Therefore, the analysis focused on anti-bacterial antibodies.

TABLE 1
Clinical information of the samples

| | Discovery set | | | Validation set | | |
|---|---|---|---|---|---|---|
| | CD | UC | HC | CD | UC | HC |
| N | 50 | 50 | 50 | 50 | 50 | 50 |
| Gender (female, male) | 29, 21 | 29, 21 | 29, 21 | 28, 22 | 28, 22 | 28, 22 |
| Age (median ± SD) | 41 ± 17.66 | 44 ± 17.25 | 42 ± 18.47 | 39.5 ± 17.49 | 44.5 ± 17.23 | 39.5 ± 16.02 |
| Disease behavior (B1/B2/B3) | 9/10/6 | | | 16/8/2 | | |
| Disease location (L1/L2/L3/L4) | 12/6/7/0 | | | 12/7/7/0 | | |
| Disease extent (E1/E2/E3) | | 0/32/18 | | | 0/34/16 | |
| Surgery (Yes, No) | 24, 25 | 8, 42 | | 22, 27 | 7, 42 | |

Fischer's exact test P value is equal to 1 for the gender difference among CD, UC and HC in both discovery and validation set. Kruskal-Wallis test P value for the age difference among CD, UC and HC in discovery and validation set were 0.3159 and 0.1737 respectively. CD: Crohn's disease; UC: Ulcerative colitis; HC: Healthy control.

TABLE 2
Bacteria and viruses studied on the microbial protein arrays.

| | Strain | Phylum | Number of proteins |
|---|---|---|---|
| Bacteria | Helicobacter pylori | Proteobacteria | 171 |
| | Staphylococcus aureus | Firmicutes | 101 |
| | Pseudomonas aeruginosa | Proteobacteria | 69 |
| | Fusobacterium varium | Fusobacteria | 58 |
| | Mycobacterium avium | Actinobacteria | 57 |
| | Streptococcus gallolyticus | Firmicutes | 46 |
| | Streptococcus pneumoniae | Firmicutes | 46 |
| | Fusobacterium nucleatum | Fusobacteria | 42 |
| | Acinetobacter calcoaceticus | Proteobacteria | 38 |
| | Phocaeicola vulgatus | Bacteroidetes | 36 |
| | Klebsiella oxytoca | Proteobacteria | 33 |
| | Acinetobacter baumannii | Proteobacteria | 27 |
| | Haemophilus influenzae | Proteobacteria | 24 |
| | Proteus mirabilis | Proteobacteria | 23 |
| | Streptococcus pyogenes | Firmicutes | 23 |
| | Lactiplantibacillus plantarum | Firmicutes | 22 |
| | Bacillus anthracis | Firmicutes | 21 |
| | Escherichia coli | Proteobacteria | 20 |
| | Shigella flexneri | Proteobacteria | 19 |
| | Citrobacter koseri | Proteobacteria | 19 |
| | Roseburia intestinalis | Firmicutes | 18 |
| | Bacteroides fragilis | Bacteroidetes | 16 |
| | Neisseria meningitidis | Proteobacteria | 16 |
| | Gemella haemolysans | Firmicutes | 15 |
| | Cutibacterium granulosum | Actinobacteria | 14 |
| | Desulfovibrio desulfuricans | Proteobacteria | 13 |
| | Vibrio cholerae | Proteobacteria | 13 |
| | Parvimonas micra | Firmicutes | 12 |
| | Enterococcus faecalis | Firmicutes | 12 |
| | Porphyromonas gingivalis | Bacteroidetes | 12 |
| | Veillonella parvula | Firmicutes | 11 |
| | Clostridioides difficile | Firmicutes | 11 |
| | Corynebacterium tuberculostearicum | Actinobacteria | 11 |
| | Streptococcus agalactiae | Firmicutes | 10 |
| | Klebsiella pneumoniae | Proteobacteria | 10 |
| | Akkermansia muciniphila | Verrucomicrobia | 10 |
| | Mycobacterium tuberculosis | Actinobacteria | 9 |
| | Campylobacter jejuni | Proteobacteria | 8 |
| | Eubacterium rectale | Firmicutes | 8 |
| | Ruminococcus albus | Firmicutes | 8 |
| | Prevotella copri | Bacteroidetes | 8 |
| | Leptotrichia buccalis | Fusobacteria | 7 |
| | Dorea formicigenerans | Firmicutes | 5 |
| | Alloiococcus otitis | Firmicutes | 5 |
| | Anaerococcus prevotii | Firmicutes | 4 |
| | Peptostreptococcus anaerobius | Firmicutes | 4 |
| | Bifidobacterium adolescentis | Actinobacteria | 3 |
| | Faecalibacterium prausnitzii | Firmicutes | 2 |
| | Collinsella aerofaciens | Actinobacteria | 2 |
| | Lachnospiraceae bacterium A4 | Firmicutes | 1 |

II. Antibodies Distinguishing CD from Healthy Controls

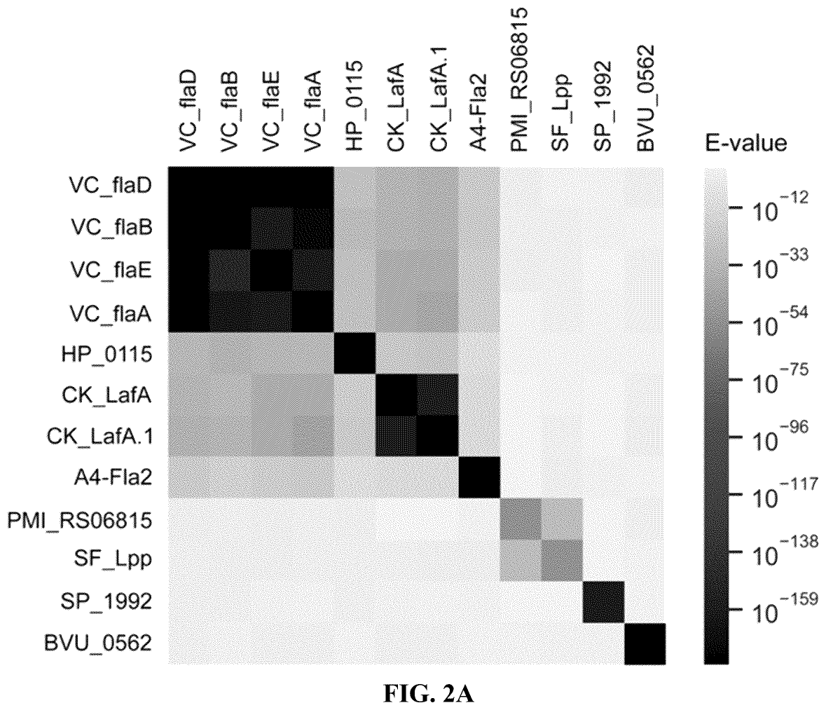

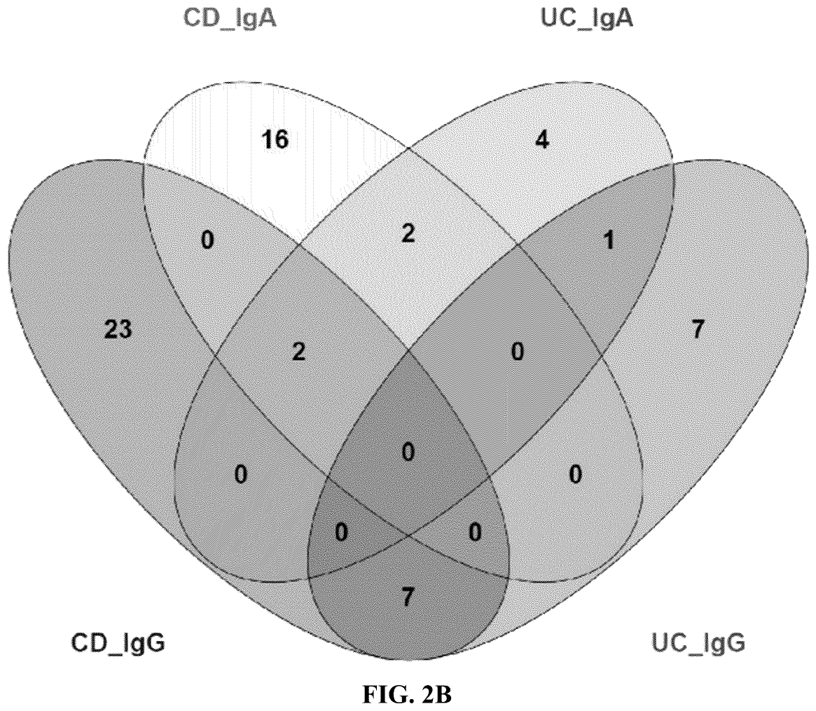

The prevalence for individual anti-microbial antibodies between CD patients and healthy controls were compared. Samples were randomly and evenly split into discovery and the validation sets (Table 1). For antibodies with elevated prevalence in CD patients, 13 IgG antibodies passed the criteria (sensitivity ≥14% at 96% specificity) in both discovery and validation sets (Table 3). Anti-A4-Fla2 IgG, a well-studied anti-bacterial flagellin antibody in CD, had the best performance with 47% sensitivity at 96% specificity in the full sample set (Table 3). Beside the flagellins, antibodies to four novel target antigens from B. vulgatus (BVU_0562), P. mirabilis (PMI_RS06815), S. flexneri (SF_Lpp) and S. pneumoniae (SP_1992) (Table 3) were found with no significant sequence homology to flagellins (FIG. 2A).

Surprisingly, 12 validated IgG antibodies showed elevated prevalence in healthy controls relative to CD patients (Table 4). Among these 12 antibodies, anti-bacterial antibodies performed better in differentiating CD patients from healthy controls than anti-viral antibodies (Table 4). Antibody against SPy_2009, an anchoring protein located in the cell wall of Streptococcus pyogenes (S. pyogenes), had the highest sensitivity of 24% at 96% specificity in healthy controls relative to CD patients. Seven validated IgA antibodies showed higher prevalence in healthy controls relative to CD patients (Table 6).

TABLE 3
Sensitivities of validated IgG antibodies comparing Crohn's disease and ulcerative colitis
with healthy controls in the discovery, validation, and the entire set at 96% specificity.

| | | Antigen | Protein name | Organism | Discovery | Validation | Entire |
|---|---|---|---|---|---|---|---|
| Crohn's | Bacteria | HP_0115 | Flagellin B | H. pylori | 28 | 48 | 38 |
| disease | | BVU_0562 | Uncharacterized protein | B. vulgatus | 26 | 22 | 25 |
| | | CK_LafA | Lateral flagellin | C. koseri | 20 | 22 | 21 |
| | | CK_LafA.1 | Lateral flagellin | C. koseri | 16 | 26 | 24 |
| | | A4-Fla2 | Flagellin | L. bacterium A4 | 40 | 54 | 47 |
| | | PMI_RS06815 | Hypothetical protein | P. mirabilis | 14 | 16 | 15 |
| | | VC_flaD | Flagellin | V. cholerae | 24 | 18 | 19 |
| | | VC_flaB | Flagellin | V. cholerae | 28 | 22 | 24 |
| | | VC_flaE | Flagellin | V. cholerae | 26 | 28 | 23 |
| | | VC_flaA | Flagellin | V. cholerae | 20 | 22 | 21 |
| | | SF_Lpp | Outer membrane | S. flexneri | 14 | 18 | 14 |
| | | | lipoprotein | | | | |
| | | SP_1992 | Cell wall surface anchor | S. pneumoniae | 20 | 16 | 18 |
| | Virus | BILF2 | Glycoprotein BILF2 | Human herpesvirus 4 | 18 | 18 | 18 |
| Ulcerative | Bacteria | CK_flgG | Flagellar basal-body rod | C. koseri | 14 | 16 | 15 |
| colitis | | | protein | | | | |
| | | A4-Fla2 | Flagellin | L. bacterium A4 | 22 | 16 | 18 |
| | Virus | BVRF2 | Capsid scaffolding | Human herpesvirus 4 | 14 | 16 | 14 |
| | | | protein | | | | |
| | | UL139 | Membrane glycoprotein | Human herpesvirus 5 | 14 | 20 | 17 |
| | | | UL139 | | | | |

TABLE 4
Sensitivities in discovery, validation, and the entire set at 96% specificity for validated
IgG antibodies with higher prevalence in healthy controls relative to CD patients.

| | Antigen | Protein name | Organism | Discovery | Validation | Entire |
|---|---|---|---|---|---|---|
| Bacteria | HP_1564 | ABC transporter substrate- | H. pylori | 14 | 16 | 16 |
| | | binding protein | | | | |
| | SACOL0985 | MAP domain-containing | S. aureus | 16 | 14 | 14 |
| | | protein | | | | |
| | AUO97_RS08350 | hypothetical protein | A. baumannii | 14 | 14 | 13 |
| | SACOL1164 | complement convertase | S. aureus | 14 | 16 | 14 |
| | | inhibitor Ecb | | | | |
| | | LPXTG-anchored | | | | |
| | SPy_2009 | fibronectin-binding protein | S. pyogenes | 38 | 22 | 24 |
| | | FbpA | | | | |
| | HI_0162 | hypothetical protein | H. influenzae | 18 | 16 | 16 |
| | PA_exoT | T3SS effector bifunctional | P. aeruginosa | 14 | 14 | 13 |
| | | cytotoxin exoenzyme T | | | | |
| | AB185_RS23245 | type VI secretion system | K. oxytoca | 18 | 18 | 19 |
| | | effector Hcp | | | | |
| | AB185_RS19385 | Hcp family type VI | K. oxytoca | 20 | 16 | 19 |
| | | secretion system effector | | | | |
| Virus | null | capsid protein, partial | Rhinovirus B14 | 14 | 22 | 17 |
| | null | nucleocapsid protein | Human | 32 | 16 | 18 |
| | | | coronavirus | | | |

III. Antibodies Distinguishing UC from Healthy Controls

For anti-microbial antibodies with elevated prevalence in UC patients relative to healthy controls, 4 IgG antibodies passed the criteria in both discovery and validation sets (Table 3). Antibodies to A4-Fla2 IgG and a flagellin from C. koseri had a sensitivity of 18% and 15% respectively. For IgG antibodies with higher prevalence in healthy controls relative to UC patients, 32 antibodies got validated (Table 5). Source microorganisms for the target antigens of these 32 antibodies were enriched for S. pneumoniae, S. aureus, and H. influenzae (2-sample proportion test, P<0.05). 2.7% of the proteins on the microbial protein microarray were from S. pneumoniae while 18.7% of antigens for validated antibodies were from S. pneumoniae, 6.1% of the proteins on the microarrays were from S. aureus while 18.7% of antigens for validated antibodies were from S. aureus, and 1.4% of the proteins on the microarrays were from H. influenzae while 12.5% of antigens for validated antibodies were from H. influenzae. Nine validated IgA antibodies showed higher prevalence in healthy controls relative to UC patients (Table 6).

Fewer anti-viral antibodies than anti-bacterial antibodies were validated comparing CD or UC patients with healthy controls (Table 3, Table 4 and Table 5). Anti-viral antibodies to Rhinovirus B14, Enterovirus C, Influenza A virus, Human metapneumovirus had higher prevalence in healthy controls compared with CD and UC patients (Tables 4 and 5).

TABLE 5
Sensitivities in discovery, validation, and the entire set at 96% specificity for validated
IgG antibodies with higher prevalence in healthy controls relative to UC patients.

| | Antigen | Protein name | Organism | Discovery | Validation | Entire |
|---|---|---|---|---|---|---|
| Bacteria | SACOL0858 | extracellular matrix protein-binding | S. aureus | 20 | 14 | 11 |
| | | adhesin Emp | | | | |
| | SACOL1140 | LPXTG-anchored heme-scavenging | S. aureus | 16 | 20 | 16 |
| | | protein IsdA | | | | |
| | SACOL0078 | phosphatidylinositol-specific | S. aureus | 14 | 20 | 16 |
| | | phospholipase C | | | | |
| | SACOL2197 | MAP domain-containing protein | S. aureus | 16 | 16 | 14 |
| | PMI_RS02875 | peptidoglycan-associated lipoprotein | P. mirabilis | 14 | 16 | 15 |
| | | Pal | | | | |
| | SPy_2191 | lytic transglycosylase domain- | S. pyogenes | 16 | 22 | 20 |
| | | containing protein | | | | |
| | SPy_cfa | CAMP factor pore-forming toxin | S. pyogenes | 18 | 20 | 16 |
| | | Cfa | | | | |
| | HI_0256 | outer membrane protein assembly | H. influenzae | 18 | 18 | 15 |
| | | factor BamC | | | | |
| | HI_null | cell envelope integrity protein TolA | H. influenzae | 14 | 18 | 15 |
| | HI_0162 | hypothetical protein | H. influenzae | 16 | 20 | 18 |
| | HI_1174 | outer membrane beta-barrel protein | H. influenzae | 16 | 16 | 16 |
| | PM_null | InlB B-repeat-containing protein | P. micra | 14 | 20 | 14 |
| | SP_1732 | Stk1 family PASTA domain- | S. pneumoniae | 24 | 32 | 24 |
| | | containing Ser/Thr kinase | | | | |
| | SP_2136 | choline-binding protein PcpA | S. pneumoniae | 22 | 32 | 23 |
| | SP_0785 | membrane-fusion protein | S. pneumoniae | 16 | 32 | 19 |
| | SP_0366 | oligopeptide ABC transporter, | S. pneumoniae | 14 | 22 | 17 |
| | | oligopeptide-binding protein AliA | | | | |
| | SP_1923 | pneumolysin | S. pneumoniae | 32 | 34 | 33 |
| | SP_0377 | choline-binding protein CbpC | S. pneumoniae | 20 | 28 | 22 |
| | SACOL1869 | serine protease SplA | S. aureus | 16 | 18 | 16 |
| | AB185_RS27465 | peptidoglycan-associated lipoprotein | K. oxytoca | 16 | 14 | 15 |
| | | Pal | | | | |
| | SACOL2291 | CHAP domain-containing protein | S. aureus | 24 | 18 | 15 |
| Virus | PVgp1 | capsid protein VP1 | Enterovirus C | 14 | 20 | 18 |
| | null | capsid protein, partial | Rhinovirus B14 | 14 | 24 | 18 |
| | null | polyprotein | Rhinovirus B14 | 34 | 36 | 28 |
| | null | polyprotein | Coxsackievirus B4 | 22 | 24 | 18 |
| | N | Nucleoprotein | Human metapneumovirus | 14 | 28 | 18 |
| | F | fusion glycoprotein | Human metapneumovirus | 16 | 24 | 20 |
| | null | fusion protein | Human respiratory | 14 | 18 | 16 |
| | | | syncytial virus B | | | |
| | PA | Polymerase acidic protein | Influenza A virus | 26 | 20 | 23 |
| | PVgp1 | genome polyprotein | Enterovirus C | 20 | 32 | 28 |
| | NP | nucleoprotein | Influenza A virus | 14 | 20 | 17 |
| | M1 | matrix protein 1 | Influenza A virus | 22 | 32 | 25 |

TABLE 6
Sensitivities in discovery, validation, and the entire set at 96% specificity for validated IgA antibodies
with higher prevalence in healthy controls relative to CD patients (Top) and UC patients (Bottom).

| | | Antigen | Protein name | Organism | Discovery | Validation | Entire |
|---|---|---|---|---|---|---|---|
| Crohn's | Bacteria | SACOL2509 | fibronectin-binding protein FnbB | S. aureus | 28 | 18 | 17 |
| disease | | SACOL2511 | fibronectin-binding protein FnbA | S. aureus | 18 | 22 | 19 |
| | | SACOL2476 | staphylopine-dependent metal ABC | S. aureus | 18 | 14 | 12 |
| | | | transporter substrate-binding protein | | | | |
| | | | CntA | | | | |
| | | SPy_2009 | LPXTG-anchored fibronectin- | S. pyogenes | 30 | 20 | 21 |
| | | | binding protein FbpA | | | | |
| | | HI_null | cell envelope integrity protein TolA | H. influenzae | 18 | 18 | 17 |
| | | HI_oapA | opacity-associated protein OapA | H. influenzae | 16 | 14 | 15 |
| | | SP_1479 | polysaccharide deacetylase family | S. pneumoniae | 18 | 20 | 20 |
| | | | protein | | | | |
| Ulcerative | Bacteria | SACOL1868 | serine protease SplB | S. aureus | 18 | 14 | 13 |
| colitis | | SACOL2509 | fibronectin-binding protein FnbB | S. aureus | 22 | 18 | 18 |
| | | HI_oapA | opacity-associated protein OapA | H. influenzae | 14 | 18 | 17 |
| | | SP_0366 | oligopeptide ABC transporter, | S. pneumoniae | 16 | 16 | 13 |
| | | | oligopeptide-binding protein AliA | | | | |
| | | SP_0346 | capsular polysaccharide biosynthesis | S. pneumoniae | 20 | 16 | 18 |
| | | | protein Cps4A | | | | |
| | | SP_0336 | penicillin-binding protein 2X | S. pneumoniae | 14 | 16 | 15 |
| | | SP_1479 | polysaccharide deacetylase family | S. pneumoniae | 18 | 18 | 14 |
| | | | protein | | | | |
| | | SP_0377 | choline-binding protein CbpC | S. pneumoniae | 20 | 16 | 18 |
| | | SACOL2194 | hyaluronate lyase HysA | S. aureus | 20 | 18 | 19 |

### IV. Comparison of Anti-Microbial Antibody Response Between CD and UC

46 IgG and 22 IgA validated anti-microbial antibodies with higher prevalence in CD patients compared to UC patients were found, while 28 IgG and 9 IgA validated anti-microbial antibodies with higher prevalence in UC patients compared to CD patients were found. There was minimal overlap of the target antigens of these validated IgG and IgA antibodies (FIG. 2B).

### V. Multivariate Analysis to Distinguish CD, UC, and Healthy Controls

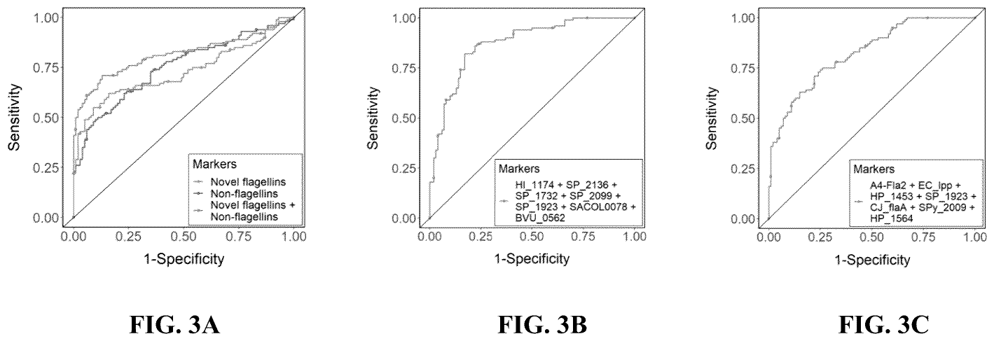

A multi-antibody panels that could distinguish CD vs control, UC vs control, and CD vs UC with an area under the curve (AUC) of 0.81, 0.87, and 0.82 respectively was built. For CD vs control, antibodies against novel flagellins (HP_0115, CK_LafA, CK_LafA.1, VC_flaD, VC_flaB, VC_flaE, VC_flaA) had an AUC of 0.73, antibodies against non-flagellins (BVU_0562, SP_1992, PMI_RS06815, and SF_Lpp) had an AUC of 0.75 and the combined AUC of antibodies against novel flagellins and non-flagellins was 0.81 (FIG. 3A). For UC vs control, a combination of seven antibodies, four against S. pneumoniae and one each against S. aureus, H. influenzae and B. vulgatus had an AUC of 0.87 (FIG. 3B). For CD vs UC, combination of seven antibodies, two against H. pylori and one each against E. coli, S. pneumoniae, S. pyogenes, C. jejuni and L. bacterium A4 had an AUC of 0.82 (FIG. 3C).

### VI. Subgroup Analysis

The association of CD behavior (B1, B2, B3), CD location (L1, L2, L3), and UC extent (E1, E2, E3) were investigated based on the Montreal classification with the anti-microbial antibody prevalence. Fourth quartile odds ratio were calculated for each antibody between the two classification groups and compared the number of antibodies with significant odds ratio (P value <0.05) in each group. B3 (penetrating) had the highest prevalence of antibodies followed by B2 (stricturing) and B1 (non-stricturing, non-penetrating) (Table 7). For CD location, L2 had the highest prevalence of antibodies followed by L3 (ileocolonic) and L1 (Table 7). For UC extent, E3 (extensive UC) had higher prevalence of antibodies compared to E2 (left sided UC). In addition to the Montreal classification, subgroup analysis was also performed based on the surgery history of CD patients. Patients who had surgery possessed higher prevalence of antibodies compared to those without surgery (Table 7).

TABLE 7
Subgroup analysis of inflammatory bowel disease patients

| | | Number of | | Two |
|---|---|---|---|---|
| | | antibodies with | | sample pro- |
| Classification | Comparison | OR > 1 | OR < 1 | portion test |
| Disease behavior | B1 vs B2 (P < 0.05) | 0 | 32 | P < 0.001 |
| B1: non- | B2 vs B3 (P < 0.05) | 0 | 19 | P < 0.001 |
| stricturing, | | | | |
| non-penetrating | | | | |
| B2: stricturing | B1 vs B3 (P < 0.05) | 2 | 41 | P < 0.001 |
| B3: penetrating | | | | |
| Disease location | | | | |
| L1: ileal | L1 vs L2 (P < 0.05) | 0 | 38 | P < 0.001 |
| L2: colonic | L2 vs L3 (P < 0.05) | 9 | 5 | P = 0.131 |
| L3: ileocolonic | L1 vs L3 (P < 0.05) | 5 | 16 | P < 0.001 |
| Disease extent | | | | |
| E2: left sided UC; | E2 vs E3 (P < 0.05) | 11 | 39 | P < 0.001 |
| E3: extensive UC | | | | |
| Surgery in CD | No vs Yes | 6 | 25 | P < 0.001 |
| patients | (P < 0.05) | | | |

For each comparison, the number of antibodies with significant difference in prevalence between two classifications were counted based on odds ratio (OR)>1 and OR<1. The difference in total number of antibodies for each comparison were computed using two sample proportion test. CD: Crohn's disease; UC: Ulcerative colitis.

### VII. Correlation of Anti-Microbial Antibodies and Autoantibodies in CD Patients

Novel autoantibodies in CD patients using the same set of CD patients and healthy controls have been previously reported. Both IgG and IgA autoantibodies and anti-microbial antibodies were profiled in all 100 CD and 100 healthy controls. It is interesting to note the antibodies showing differences for autoantibodies were mostly IgA, but the anti-microbial antibodies were mostly IgG. Anti-SNRPB_IgA had the highest sensitivity of 20% at 96% specificity among all autoantibodies compared with 47% sensitivity at 96% specificity for the best performing anti-microbial antibody, anti-A4-Fla2_IgG.

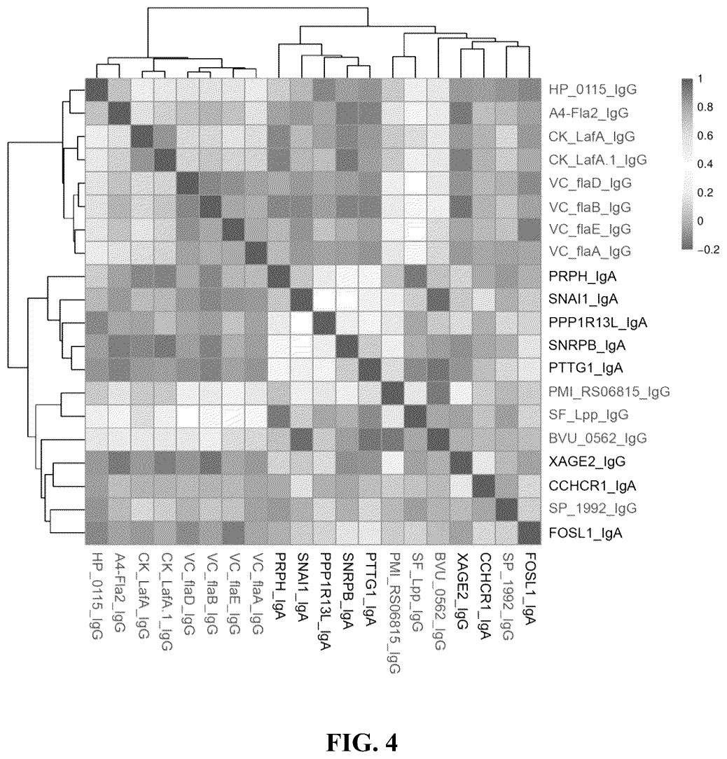

The novel autoantibodies and validated anti-microbial antibody profiles were compared to determine if correlation existed between their reactivity. Overall, high correlation between autoantibodies and anti-microbial antibodies in CD patients was not observed (FIG. 4). Anti-microbial antibodies formed two clusters, one with anti-flagellin antibodies, and the other with SF_Lpp IgG and PMI_RS06815 IgG. Five autoantibodies, PRPH_IgA, SNAI1_IgA, PPP1R13L_IgA, SNRPB_IgA and PTTG1_IgA, formed a cluster. The remaining antibodies had relatively unique reactivity patterns.

### VIII. Materials and Methods

a. Patients and Samples

All the serum samples were acquired from Serum Biobank at Mayo Clinic with approval from institutional review board. CD patients were randomly selected, followed by age and gender matched healthy controls and UC patients. The samples (100 CD, 100 UC and 100 controls) were divided evenly into two non-overlapping discovery and validation sets randomly (Table 1). Disease status for study participants was assessed by clinicians at Mayo clinic.

b. Microbial Protein Array Fabrication

Of the 1570 microbial proteins analyzed, 1173 proteins were from 50 different species of bacteria, 397 proteins were from 33 different species of viruses and the remaining proteins were autoantigens. These proteins were selected from a large collection of microbial antigens (DNASU.org) with reference to the anti-microbial antibody studies on other diseases (unpublished data). Microbial protein arrays were fabricated as described earlier. Briefly, plasmids with genes of interest cloned in the pANT7_cGST expression vector were obtained from the DNASU plasmid repository, prepared, and printed into silicon nanowells using a piezoelectric dispensing system to produce microbial protein arrays. On the day of experiment, proteins were freshly expressed from printed plasmids using an in-vitro transcription and translation protein expression kit (Fisher Scientific) and captured by anti-GST antibody co-printed in each nanowell. After expression, microarrays were incubated with 1:100 diluted serum samples. The case and control serum samples were randomized while profiling on microarray to reduce bias. IgG and IgA anti-microbial protein antibodies were detected by Alexa-647 goat anti-human IgG (H+L) and Cy3 goat anti-human IgA (Jackson ImmunoResearch). After washing and drying, the microarrays were scanned in a Tecan PowerScanner and the raw fluorescence intensity data were extracted using the ArrayPro Analyzer Software. Raw fluorescence intensity of each protein on the microarray was divided by the median intensity of all the proteins on the microarray for normalization. The normalized value was termed as Median Normalized Intensity (MNI) and used for all analysis. Seropositivity of antibody for a particular antigen was defined as MNI≥2 as have been done for other studies.

c. Statistical Analysis

Pairwise comparisons of numbers of IgG or IgA antibodies for each bacterial species among the 3 subject groups were performed using Chi-squared tests to assess statistical significance (FIG. 5). For each pairwise comparison, the Chi-squared P values were adjusted using the FDR (false discovery rate) method to reduce the likelihood of false positives. In addition to the multiple comparison adjustment at the antibody level, adjustment was performed at the species level.

For univariate analysis between two comparison groups, sensitivity was calculated for one group at the 96th percentile of the other group or the MNI of 2, whichever was larger. Antibodies with ≥14% sensitivity in the discovery set were selected as candidates for further validation. If an antibody had ≥14% sensitivity at 96% specificity in both discovery and validation sets, then it was considered as a “validated marker. Venn diagram for the overlap of microbial antigen targets were plotted using Venny.

A three-stage approach was used to build the multi-antibody panels. In the first stage, all candidate biomarkers that passed the criteria above, i.e., sensitivity was greater or equal than 14% at 96% specificity were selected. Next, the minimum redundancy maximum relevance algorithm was applied to further select biomarkers that were possibly the most important and least correlated. In the third stage, a logistic regression model was used to fit the selected biomarkers from the first two stages and generated its receiver operating characteristic curve and AUC value to evaluate the model's discriminatory performance between CD, UC, and healthy controls.

Pair-wise subgroup comparisons based on the Montreal classification were performed for the odds ratio (OR) of each antibody using the seropositivity threshold defined as the maximum of MNI 2 and the 75th percentile of all samples. Chi-squared tests were used to test global significance between all groups with a slight modification by adding 0.5 to each cell of the table to avoid zero cell counts. P values from the chi-squared method were adjusted for each pair of comparisons and for all candidate biomarkers. The number of antibodies with significant difference in prevalence among classifications were counted based on OR>1 and OR<1 for each pair of classification of CD behavior, CD location, UC extent and the surgery history of CD patients. The difference in total number of antibodies with significant difference between classification groups were computed using two sample proportion test. A subgroup analysis for the UC patients based on the surgery history because most (84 out of 100) had no surgeries (Table 1).

Spearman's rank correlation analysis was performed to assess the correlation between autoantibody and anti-microbial antibody reactivity for CD patients and healthy controls. The R “pheatmap” package was used to generate the heatmap for correlation coefficients.

d. Bioinformatics Analysis

The NCBI Taxonomy browser was used to find the taxonomical details of all the bacteria and viruses used in the study. The taxa were downloaded as phylip tree file and was used as an input in interactive tree of life software. Two phylogenetic trees were created for bacteria and viruses with different colors distinguishing the phylum.

For sequence homology analysis, a pair-wise BLAST analysis was carried out on the antigen protein sequences of validated antibodies for CD vs healthy control analysis. E-values were used to generate a heatmap using Python Seaborn package.

What is claimed is:

1. An antibody panel for diagnosing a subject with inflammatory bowel disease, the antibody panel comprising at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, SP_1992, BILF2, CK_flgG, A4-Fla2, BVRF2, and UL139.

2. The antibody panel of claim 1, wherein the inflammatory bowel disease is Crohn's disease, the antibody panel comprises at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2.

3. The antibody panel of claim 2, wherein the antibody panel comprises HP_0115, CK_LafA, CK_LafA.1, VC_flaD, VC_flaB, VC_flaE, and VC_flaA.

4. The antibody panel of claim 2, wherein the antibody panel comprises BVU_0562, SP_1992, PMI_RS06815, and SF_Lpp.

5. The antibody panel of claim 2, wherein the antibody panel comprises HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2.

6. The antibody panel of claim 1, wherein the inflammatory bowel disease is ulcerative colitis, the antibody panel comprises at least at least one antigen selected from the group consisting of: CK_flgG, A4-Fla2, BVRF2, and UL139.

7. The antibody panel of claim 6, wherein the antibody panel comprises CK_flgG, A4-Fla2, BVRF2, and UL139.

8. A method of diagnosing inflammatory bowel disease in a subject with gastrointestinal distress, the method comprising:
providing a biofluid sample from the subject with gastrointestinal distress;
contacting the biofluid sample with at least one antigen selected from the group consisting of: HP_0115, BVU_0562, CK_LafA, CK_LafA.1, A4-Fla2, PMI_RS06815, VC_flaD, VC_flaB, VC_flaE, VC_flaA, SF_Lpp, SP_1992, BILF2, CK_flgG, A4-Fla2, BVRF2, and UL139; and
determining if the biofluid sample comprises an antibody against the at least one antigen, wherein the presence of the antibody against the at least one antigen diagnoses the subject with gastrointestinal distress with an inflammatory bowel disease.

9. The method of claim 8, wherein the biofluid sample is contacted with HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2, wherein the presence of antibodies against HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2 diagnoses the subject with gastrointestinal distress with Crohn's disease.

10. The method of claim 9, wherein the biofluid sample is in contact with HP_0115, CK_LafA, CK_LafA.1, VC_flaD, VC_flaB, VC_flaE, and VC_flaA, wherein the presence of HP_0115, CK_LafA, CK_LafA.1, VC_flaD, VC_flaB, VC_flaE, and VC_flaA diagnoses the subject with gastrointestinal distress with Crohn's disease instead of ulcerative colitis.

11. The method of claim 9, wherein the antibody panel comprises BVU_0562, SP_1992, PMI_RS06815, and SF_Lpp, wherein the presence of BVU_0562, SP_1992, PMI_RS06815, and SF_Lpp diagnoses the subject with gastrointestinal distress with Crohn's disease instead of ulcerative colitis.

12. The method of claim 9, wherein the antibody panel comprises HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2, wherein the presence of HP_0115, BVU_0562, CK_LafA, CK_LafA.1, A4-Fla2, PMI_RS06815, VC_flaD, VC_flaB, VC_flaE, VC_flaA, SF_Lpp, SP_1992, and BILF2 diagnoses the subject with gastrointestinal distress with Crohn's disease instead of ulcerative colitis.

13. The method of claim 8, wherein the biofluid sample is contacted with at least one of CK_flgG, A4-Fla2, BVRF2, and UL139, wherein the presence of antibodies against CK_flgG, A4-Fla2, BVRF2, and UL139 diagnoses the subject with gastrointestinal distress with ulcerative colitis.

14. The method of claim 13, wherein the biofluid sample is contacted with CK_flgG, A4-Fla2, BVRF2, and UL139.

15. The method of claim 8, wherein the biofluid sample is blood or serum.

16. A method of distinguishing the cause of gastrointestinal distress in a subject, the method comprising:
providing a biofluid sample from a subject with gastrointestinal distress;
contacting the biofluid sample with at least one antigen selected from the group consisting of: SACOL2509, SACOL2511, SACOL2476, SPy_2009, HI_null, HI_oapA, SP_1479, SACOL1868, SACOL2509, HI_oapA, SP_0366, SP_0346, SP_0336, SP_1479, SP_0377, and SACOL2194; and
determining the biofluid sample comprises an antibody against the at least one antigen, wherein the presence of the antibody against the at least one antigen diagnoses the subject with gastrointestinal distress with an inflammatory bowel disease.

17. The method of claim 16, wherein the biofluid sample is contact with SACOL2509, SACOL2511, SACOL2476, SPy_2009, HI_null, HI_oapA, and SP_1479, the presence of antibodies against SACOL2509, SACOL2511, SACOL2476, SPy_2009, HI_null, HI_oapA, and SP_1479 diagnoses the subject with gastrointestinal distress with Crohn's disease instead of ulcerative colitis.

18. The method of claim 16, wherein the biofluid sample is contact with SACOL1868, SACOL2509, HI_oapA, SP_0366, SP_0346, SP_0336, SP_1479, SP_0377, and SACOL2194, the presence of antibodies against SACOL1868, SACOL2509, HI_oapA, SP_0366, SP_0346, SP_0336, SP_1479, SP_0377, and SACOL2194 diagnoses the subject with gastrointestinal distress with ulcerative colitis instead of Crohn's disease.

19. The method of claim 16, wherein the biofluid sample is blood or serum.

20. The method of claim 16, wherein the biofluid sample is blood.
